(19) United States
(12) Patent Application Publication
(10) Pub. No.: US 2010/0273712 A1
(43) Pub. Date: Oct. 28, 2010

(54) SURFACTANT USAGE IN LUNG TRANSPLANTATION AND METHODS THEREOF

(76) Inventor: Edmund A. Egan, Amherst, NY (US)

Correspondence Address: Paul T. Lavoie;Jaeckle Fleischmann &Mugel, LLP, 12 Fountain Plaza, Buffalo, NY, 14202-2292, US

(21) Appl. No.: 12/623,937

(22) Filed: Nov. 23, 2009

(30) Foreign Application Priority Data
May 23, 2008 (US) PCT/US2008/064785

(51) Int. Cl. A61K 38/16; A61P 11/00

(52) U.S. Cl. 514/15.5

(57) ABSTRACT

The present invention is directed to the use of pulmonary surfactants, and particularly pulmonary surfactants containing hydrophobic surfactant-associated proteins B, C or both (i.e., surfactants such as calfactant (Infasurf®), to treat PGD and other adverse effects of lung transplantation.

### BACKGROUND OF THE INVENTION

### Cross-Reference to Related Applications

The present applicant claims the benefit of U.S. Provisional Application No. 60/939,996 filed on May 24, 2007, which application is incorporated herein in its entirety by reference.

### DISCUSSION OF THE BACKGROUND ART

Although successful lung transplantations were first performed in the 1980s, lung transplants have become widely used only as a result of a variety of improvements in the methodologies of such procedures. Thus improvements in, for example, donor management, donated lung preservation, immunosupression methods for preventing rejection of donated lungs by the lung transplant recipient, and infection-fighting therapies (e.g., antimicrobials) for post-surgical recovery have all contributed to a rise in the number of such transplantations performed worldwide. Specifically; in 1984 only a few patients at one center received lung transplants; by contrast, over 1,500 patients at about 100 centers were receiving transplants by the mid-1990s.

Despite the general successfulness of lung transplantation methods, there is still a variety of serious and, in some cases, lethal consequences of these procedures. Thus for example ischemia/reperfusion (I/R) injury to transplanted lung tissue resulting from the loss and then subsequent restoration of blood supply to that tissue can result in the delayed withdrawal of patient ventilator support in roughly 30% of transplant recipients. In about 10% of recipients, I/R injury is severe enough to produce what is termed “primary graft failure” (PGF) or, synonymously, “primary graft dysfunction” (PGD), which is associated with extremely serious or even lethal patient outcomes such as significantly prolonged patient hospitalization in an intensive care unit, and 20 to 30% increases in 90-day patient mortality rates. The negative consequences of PGD for patient survival can perhaps best be described by noting that PGD is responsible for about 30% of the patient deaths occurring within 30 days post-transplantation.

Unfortunately, despite these serious or even lethal effects of PGD, to date there is no adequate method of treating PGD. Thus as stated in a 2006 review of Lung Transplantation (Pierson, “Lung Transplantation: Current Status and Challenges”, Transplantation, 81:1609-1615 (2006)), PGD has persisted despite, e.g., the “introduction of an extracellular preservation solution specifically tailored to the lung (Perfadex®), and wide adoption of improved procurement techniques such as retrograde perfusion through the pulmonary veins.” Similarly, a variety of other potential treatment schemes have to date failed to produce any adequate treatments for PGD. For example, although cytokines, chemokines, and adhesive ligand/receptor interactions are all potential bases for drugs or other treatment methods to reduce adverse outcomes in lung transplantation, actual therapeutic applications based on the molecules are not imminent.

Thus in light of the above, there is a real need to develop effective methods to treat PGD or other adverse effects of lung transplantation.

Moreover, there is also a need to develop methods that prevent PGD altogether, i.e., methods that provide prophylactic (preventative or protective) mechanisms for lung tissue used in transplantation.

### SUMMARY OF THE INVENTION

The present invention is directed to the use of pulmonary surfactants, and particularly pulmonary surfactants containing hydrophobic surfactant associated proteins B or C (SP-B or SP-C) or both (i.e., surfactants such as calfactant (Infasurf®), to treat PGD and other adverse effects of lung transplantation. The present invention is also directed to the use of surfactants prophylatically, i.e., to reduce the incidence of PGD or other adverse effects associated with lung transplantation, or even to entirely prevent PGD or other adverse effects associated with lung transplantation.

Thus in one aspect, the present invention is directed to a method of preventing primary graft dysfunction in a lung transplantation patient, comprising administering a therapeutically effective dosage of a surfactant to the patient. In one embodiment, the surfactant contains detectable SP-B activity. In another embodiment, the surfactant is calfactant.

In one embodiment of the invention, there is a method of reducing the risk or seriousness of primary graft dysfunction in a lung transplantation patient. The method comprises the step of administering a therapeutically effective dosage of a surfactant to a patient. Typically, the surfactant is administered after the patient receives a lung transplant. The administration preferably occurs before re-expansion of the lung and ventilation of the donated lungs.

In one embodiment, the surfactant is administered by an injection of the surfactant into the trachea through the wall of a trachea.

In another embodiment, the surfactant is administered by inhalation of the surfactant into the lungs with an inhalation device such as a nebulizer or an inhaler.

In still another embodiment, the patient does not develop Grade 3 primary graft dysfunction.

In an embodiment, the surfactant is calfactant.

In another embodiment, the surfactant formulation comprises SP-B phospholipid in a carrier, and the amount of SP-B is a minimum of 0.01 wt. %, 0.05 wt. %, 0.75 wt. %, 1.5 wt. %, 2.0 wt. % and a maximum of 4.0 wt. %, 3.5 wt. %, 3.0 wt. %, 2.5 wt. %, 2.0 wt. % based upon the total weight of the surfactant formulation.

In another embodiment, the calfactant is in the form of a suspension. It is desirable that the suspension has an active ingredient that has a detectable level of activity at the concentration present and inactive ingredients. More preferably, wherein the concentration of the active ingredient is a minimum of 20 mg, 30 mg, 40 mg of surfactant per ml of suspension and a maximum of 100 mg, 90 mg, 80 mg, 70 mg, 60 mg, 50 mg, 40 mg, or 30 mg of surfactant per ml of suspension.

In one embodiment, the surfactant is instilled into the transplanted lungs using a fiber optic bronchoscope. In another embodiment, the surfactant includes an active ingredient that is selected from the group consisting essentially of SP-B, SP-C and combinations thereof surfactant. Preferably, the surfactant is SP-B.

Optionally, the present invention includes a method of lung transplantation to prophylactically reduce the risk of primary graft dysfunction. The method comprises the step of replacing a lung in a patient with a donor lung. The method comprises administering a therapeutically effective dosage of a surfactant to the donor lung.

### BRIEF DESCRIPTION OF THE DRAWINGS

FIG. 1 presents the demographics of the patient population recruited for the lung-transplantation study discussed herein.

FIG. 2 presents details of the number of patients used for each procedure, dosing amount of the surfactant calfactant, and ischemia time.

FIG. 3 presents data on oxygenation levels from 0 to 48 hrs post transplantation.

FIG. 4 presents data on various effects of the use of surfactant versus control on patient recovery parameters.

FIG. 5 presents data demonstrating that patient long-term lung function and rejection was not influenced by the administration of the surfactant calfactant. In this figure, the standard abbreviation “FEV₁” is used to represent the serial measurement of forced expiratory volume in 1 second.

### DETAILED DESCRIPTION OF THE INVENTION

The present invention is directed to the use of pulmonary surfactants, and particularly pulmonary surfactants containing hydrophobic surfactant associated proteins B or C (SP-B or SP-C) or both (i.e., surfactants such as calfactant (Infasurf®)), to treat PGD and other adverse effects of lung transplantation. The present invention is also directed to the use of surfactants prophylactically, i.e., to reduce the incidence of PGD or other adverse effects associated with lung transplantation, or even to entirely prevent PGD or other adverse effects associated with lung transplantation.

As used herein, “pulmonary surfactant” or, synonymously, “surfactant” refers to any composition that acts to prevent lung collapse or facilitates lung aeration by its dynamic modification of alveolar surface tension during the respiratory cycle. Surfactants contemplated in the present invention indude synthetic, protein-free surfactants such as Exosurf®, as well as surfactants derived from natural sources, e.g., Curosurf®, Survanta®, and Infasurf® (synonymously, calfactant). Surfactants containing hydrophobic surfactant associated proteins B (SP-B) and/or C (SP-C) are particularly preferred, and more particularly surfactants containing active amounts of SP-B and/or SP-C. Thus for example, a surfactant contemplated herein may include 0.01 wt. % to about 4 wt. % SP-B phospholipid.

Also contemplated are surfactants containing analogs or derivatives of SP-B or/and SP-C, i.e., proteins that are either derived from SP-B, or SP-C by truncation of the amino acid sequences of these proteins, substitutions, or other means as would be well-known to the skilled artisan. Such proteins are alternately or additionally defined by their functionality. Thus such proteins may have “SP-B-like” or “SP-C-like” functionality, i.e., these proteins have one or more of the surfactant properties conferred by the naturally occurring SP-B or SP-C proteins.

In this regard, functionality or, synonymously, activity, may be measured by any technique known for measuring the activity or activities of these proteins. For example, SP-B activity may be measured as biophysical

activity, as determined by, e.g., observing that the surface tension of an inverted air “bubble” in the composition under consideration reaches <3 mN/m at minimum bubble volume within 5 minutes when oscillated in a “Pulsating Bubble Surfactometer” (Electronetics, Amherst, NY) at 20 cycles per minute. See, e.g., Wang et al., Am. J. Physiol. Lung Cell Mol. Physiol., 2831897 (2002). Activity may also be measured by biological activity, e.g., by observing restoration to normal of the deflation pressure-volume curve in an excised or in situ surfactant deficient animal lung using a method such as that of Bermel (Lung, 162:99-113 (1984)) or Mizuno (Pediatr. Res., 37:271-276 (1995)),

Although the present invention encompasses any surfactant, calfactant is particularly preferred. A range of dosing amounts of calfactant may be used, depending on the method of administration of this composition. In Example 1 presented below, a 35 mg/ml suspension of calfactant was administered at 19 ±2 mg phospholipid/kg body weight using a bronchoscope so that every lobe of the transplanted lung was treated. Although this administration amount and method are preferred, the present invention is explicitly not limited to this amount and method. Thus other amounts of calfactant (or other surfactant) may be used. For example, when represented in terms of the phospholipid component of the surfactant (when appropriate), an amount may be used of between 10 mg phospholipid/kg body weight and about 200 mg phospholipid/kg body weight. In another embodiment of the present invention, calfactant (or other surfactant) may be used as a formulation comprising a saline suspension of the calfactant (or other surfactant) at about 25 mg/ml to about 100 mg/ml of phospholipid, plus SP-B in an amount of about 0.1 wt. % to about 4.0 wt. %, based on the weight of the phospholipids.

Also contemplated are a variety of methods of administration, e.g., administration of surfactant as an inhalable formulation (e.g., nebulizer). Moreover it may be preferable to use multiple boluses of calfactant rather than a single administered dose. And a variety of methods of delivery are contemplated, including, but not limited to, delivery via catheter, etc. It should also be understood that a similar variety of methods of administration, dose, and forms of the compound to be administered are also contemplated for surfactants other than calfactant.

### Example 1: Pilot Trial Using the Surfactant Calfactant

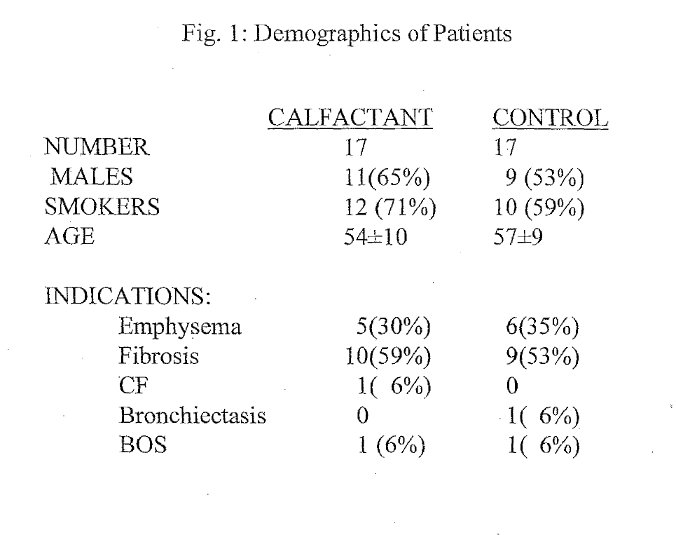

The demographics of the patient population used for a pilot trial of calfactant effect on lung transplantation are shown in FIG. 1. In most cases, the lungs from a single donor were used for two recipients where, randomly, one recipient was not treated with surfactant and the other recipient received calfactant.

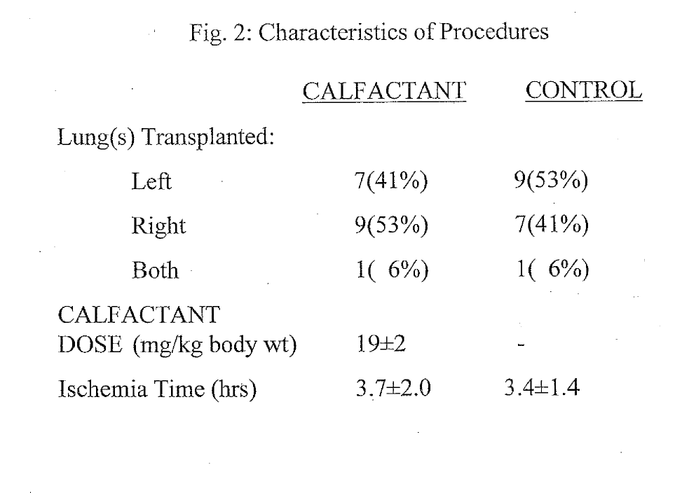

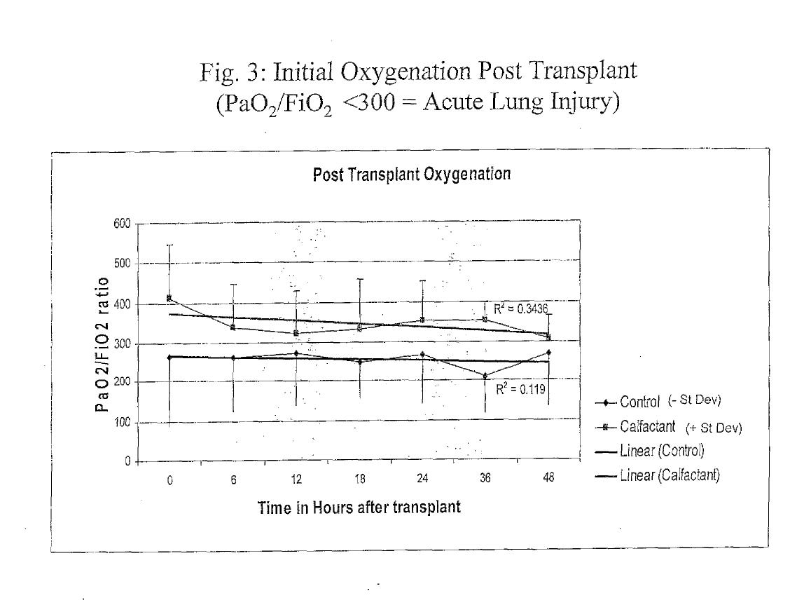

Administration of calfactant was done after lung transplantation, using a single acute dose of 19 ±2 mg phopholipid/kg body weight of calfactant suspension at a concentration of 35 mg/ml instilled into the transplanted lungs using a fiber optic bronchoscope. See FIG. 2. Oxygenation was measured continuously by transcutaneous pulse oximetry and intermittently by measurement of the partial pressure of oxygen in arterial blood. The ratio of arterial oxygen partial pressure, PaO₂, to fraction of inspired oxygen, RO₂, was measured for the first 2 days post-transplantation, as shown in FIG. 3. As this figure indicates, a PaO₂/ FiO₂ of less than 300 is indicative of acute lung injury (ALI).

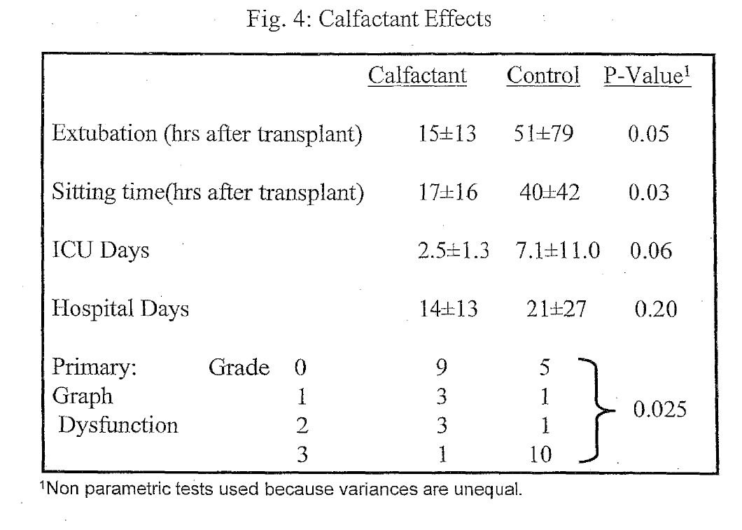

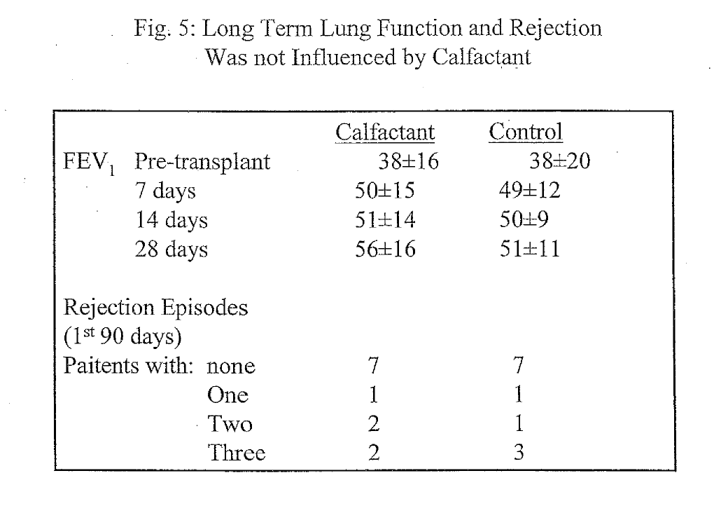

FIGS. 4 to 5 summarize the results obtained in this pilot study using calfactant versus control. The results can best be understood with reference to the following scale: A Grade 0 PGD is observed when a patient has a PaO₂/ FiO₂ that is greater than 300 and has a radiographic infiltrate that is not consistent with pulmonary edema. A Grade 1 PGD is observed when a patient has a PaO₂/ FiO₂ that is greater than 300 and has a radiographic infiltrate that is consistent with pulmonary edema. A Grade 2 PGD is observed when a patient has a PaO₂/ FiO₂ of between 200 and 300 and a radiographic infiltrate consistent with pulmonary edema. A Grade 3 PGD is observed when a patient has a PaO₂/ FiO₂ that is less than 200 and a radiographic infiltrate consistent with pulmonary edema).

Specifically, patients treated with calfactant were extubated significantly earlier than untreated patients; were able to be mobilized significantly earlier than untreated patients; and spent significantly fewer days in the intensive care unit than untreated patients. Treated patients also spent a fewer number of days, on average, in the hospital than untreated patients, although the statistical significance of this observation is not clear.

Equally as significantly, the data obtained in this pilot study demonstrate that calfactant treatment significantly lowers the risk of Grade 3 PGD, which is the most serious early complication of lung transplants. This observation is particularly, novel given that, in general, the focus in complications of lung transplantation has been on treatment of PGD, rather than its prevention. Thus, the results of this pilot experiment suggest a prophylactic use of surfactant, particularly calfactant, to prevent PGD. As noted above, this use does not preclude the use of surfactant in the treatment of PGD as well as its prevention, with this latter use being specifically contemplated in the present invention.

Finally, the data in FIG. 5 demonstrates that calfactant has no observed effects on lung term lung function or risk of rejection. This is in keeping with the administration of calfactant in this pilot study as a single acute dose. Although such a dosing regimen is desirable, the present invention explicitly contemplates multiple dosing regimens, as well as long-term administration of surfactant, etc. While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A method of reducing the risk or seriousness of primary graft dysfunction in a lung transplantation patient, comprising the step of administering a therapeutically effective dosage of a surfactant to the patient.

2. The method of claim 1, wherein the surfactant is administered after the patient receives a lung transplant.

3. The method of claim 1, wherein the surfactant is administered by an injection of the surfactant into the trachea through the wall of a trachea.

4. The method of claim 1, wherein the surfactant is administered by inhalation of the surfactant into the lungs with an inhalation device.

5. The method of claim 1, wherein the patient treated does not develop Grade 3 primary graft dysfunction.

6. The method of claim 1, wherein the surfactant is calfactant.

7. The method of claim 6, wherein the therapeutically effective dosage is at least 10 mg calfactant/kg body weight

8. The method of claim 7, wherein the calfactant is in the form of a suspension.

9. The method of claim 7, wherein the suspension has an active ingredient that has a detectable level of activity at the concentration present and inactive ingredients, wherein the concentration of the suspension is a minimum of 20 mg of surfactant per ml of suspension and a maximum of 100 mg of surfactant per ml of suspension.

10. The method of claim 1, wherein the calfactant is instilled into the transplanted lungs using a fiber optic bronchoscope.

11. The method of claim 1, wherein the surfactant is selected from the group consisting essentially of SP-A, SP-B and combinations thereof surfactant.

12. . The method of claim 1, wherein the surfactant is SP-B.

13. A method of lung transplantation to prophylactically reduce the risk of primary graft dysfunction, comprising the steps of: replacing a lung in a patient with a donor lung; and administering a therapeutically effective dosage of a surfactant to the donor lung.

14. The method of claim 13, wherein the surfactant is administered after the patient receives a lung transplant.

15. The method of claim 13, wherein the surfactant is administered by an injection of the surfactant into the trachea through the wall of a trachea.

16. The method of claim 13, wherein the surfactant is administered by inhalation of the surfactant into the lungs with an inhalation device.

17. The method of claim 13, wherein the patient treated does not develop Grade 3 primary graft dysfunction.

18. The method of claim 13, wherein the surfactant is calfactant.

19. The method of claim 18, wherein the therapeutically effective dosage is at least 10 mg calfactant/kg body weight

20. The method of claim 19, wherein the calfactant is in the form of a suspension.

21. The method of claim 19, wherein the suspension has an active ingredient that has a detectable level of activity at the concentration present and inactive ingredients, wherein the concentration of the suspension is a minimum of 20 mg of surfactant per ml of suspension and a maximum of 100 mg of surfactant per ml of suspension.

22. The method of claim 13, wherein the calfactant is instilled into the transplanted lungs using a fiber optic bronchoscope.

23. The method of claim 13, wherein the surfactant is selected from the group consisting essentially of SP-C, SP-B and combinations thereof.

24. The method of claim 13, wherein the surfactant is SP-B.

25. The method of claim 1, wherein the step of administering occurs after lung transplantation but before re-expansion and ventilation.

26. The method of claim 13, wherein the step of administering occurs after lung transplantation, but before re-expansion.
